(19) United States
(12) Patent Application Publication
(10) Pub. No.: US 2006/0240067 A1
(43) Pub. Date: Oct. 26, 2006

(54) Biocompatible articles and related methods

(76) Inventors: Scott H. Gillis, Concord, MA (US); Steve Chartier, Bedford, NH (US); Wen Gang Chen, Woburn, MA (US); Cheng Zai Lu, Newton, MA (US); Paul Schechter, Dover, MA (US)

Correspondence Address: FISH & RICHARDSON PC, P.O. BOX 1022, MINNEAPOLIS, MN, 55440-1022, US

(21) Appl. No.: 11/391,761

(22) Filed: Mar. 29, 2006

Related U.S. Application Data
(60) Provisional application No. 60/666,037, filed on Mar. 29, 2005.

(51) Int. Cl. A61K 33/38; A61F 2/00

(52) U.S. Cl. 424/423; 424/618; 977/906

(57) ABSTRACT

Biocompatible articles and related methods are disclosed.

### CROSS-REFERENCE TO RELATED APPLICATION

This application claim benefit under 35 U.S.C. §119(e)(1) of U.S. Provisional Patent Application Ser. No. 60/666,037, filed Mar. 29, 2005, and entitled “Biocompatible Articles and Related Methods”, the contents of which is hereby incorporated by reference.

### TECHNICAL FIELD

The invention relates to biocompatible articles and related methods.

### BACKGROUND

It is generally desirable to treat a subject (e.g., a human) that has an undesirable condition. Many different compositions have been developed to treat undesirable conditions. For example, certain forms of silver have been reported to be effective in treating some undesirable skin conditions.

### SUMMARY

The invention relates to biocompatible articles and related methods.

In one aspect, the invention features an article that includes a biocompatible material and an antimicrobial material associated with the biocompatible material. The biocompatible material is a naturally occurring biocompatible material.

In another aspect, the invention features an article that includes a biocompatible material and an anti-MMP material associated with the biocompatible material. The biocompatible material is a naturally occurring biocompatible material.

In a further aspect, the invention features an article that includes a biocompatible material and an anti-inflammatory material associated with the biocompatible material. The biocompatible material is a naturally occurring biocompatible material.

In another aspect, the invention features a method that includes implanting an article in or on a subject. The article includes a biocompatible material and an antimicrobial material associated with the biocompatible material. The biocompatible material is a naturally occurring biocompatible material.

In a further aspect, the invention features a method that includes using an article to bulk tissue in a subject. The article includes a biocompatible material and an antimicrobial material associated with the biocompatible material. The biocompatible material is a naturally occurring biocompatible material.

In an additional aspect, the invention features a method that includes using an article to reinforce tissue in a subject. The article includes a biocompatible material and an antimicrobial material associated with the biocompatible material. The biocompatible material is a naturally occurring biocompatible material.

In another aspect, the invention features a method that includes using an article to suspend tissue in a subject. The article includes a biocompatible material and an antimicrobial material associated with the biocompatible material. The biocompatible material is a naturally occurring biocompatible material.

In a further aspect, the invention features a method that includes combining a biocompatible material and an antimicrobial material to form an article.

In one aspect, the invention features an article that includes a biocompatible material and a metal-containing material associated with the biocompatible material. The biocompatible material is a naturally occurring biocompatible material.

In another aspect, the invention features a method that includes implanting an article in or on a subject. The article includes a biocompatible material and a metal-containing material associated with the biocompatible material. The biocompatible material is a naturally occurring biocompatible material.

In a further aspect, the invention features a method that includes using an article to bulk tissue in a subject. The article includes a biocompatible material and a metal-containing material associated with the biocompatible material. The biocompatible material is a naturally occurring biocompatible material.

In an additional aspect, the invention features a method that includes using an article to reinforce tissue in a subject. The article includes a biocompatible material and a metal-containing material associated with the biocompatible material. The biocompatible material is a naturally occurring biocompatible material.

In another aspect, the invention features a method that includes using an article to suspend tissue in a subject. The article includes a biocompatible material and a metal-containing material associated with the biocompatible material. The biocompatible material is a naturally occurring biocompatible material.

In a further aspect, the invention features a method that includes combining a biocompatible material and a metal-containing material to form an article. The biocompatible material is a naturally occurring biocompatible material.

Other features and advantages of the methods will be apparent from the description and drawings, and from the claims.

### DESCRIPTION OF DRAWINGS

FIG. 1 is a cross-sectional view of an embodiment of an article;

FIG. 2 is a cross-sectional view of an embodiment of an article; and

FIG. 3 is a cross-sectional view of an embodiment of an article.

### DETAILED DESCRIPTION

In some embodiments, the invention relates to articles that include a biocompatible material and an antimicrobial material. In general, the articles can be prepared and used as desired. In certain embodiments, the articles can be used in a subject. For example, the articles can be implants, artificial tissue (e.g., artificial synovium, artificial cartilage), tissue replacements, bone replacements, tissue bulking agents, tissue reinforcing agents, and/or tissue suspending agents.

In embodiments in which the articles are used as implant, the articles can be in the shape of, for example, a screw, a nail, a staple, a thread, a string, a catheter, a valve, a stent, a graft, a ligament replacement, a suspension reinforcement and/or an attachment. In some embodiments, the article can be a wound dressing or a wound packing.

In embodiments in which the articles are used as tissue bulking agents, tissue reinforcing agents, and/or tissue suspending agents, the articles can be used to bulk tissue in, for example, reconstructive procedures, cosmetic procedures and/or to treat certain conditions (e.g., urinary incontinence, fecal incontinence, gastroesophageal reflux disease, vesicoureteral reflux disease, vocal cord paralysis, inguinal hernia repair, ventral hernia repair, heart valve repair, heart valve replacement, anterior cruciate ligament (ACL) repair, ACL replacement, MCL repair, deviated septum repair, dental packing).

In some embodiments, the biocompatible material is in the form of a substrate (e.g., when used as an implant). In such embodiments, the antimicrobial material can be, for example, supported by (e.g., disposed on) the substrate and/or disposed in the substrate.

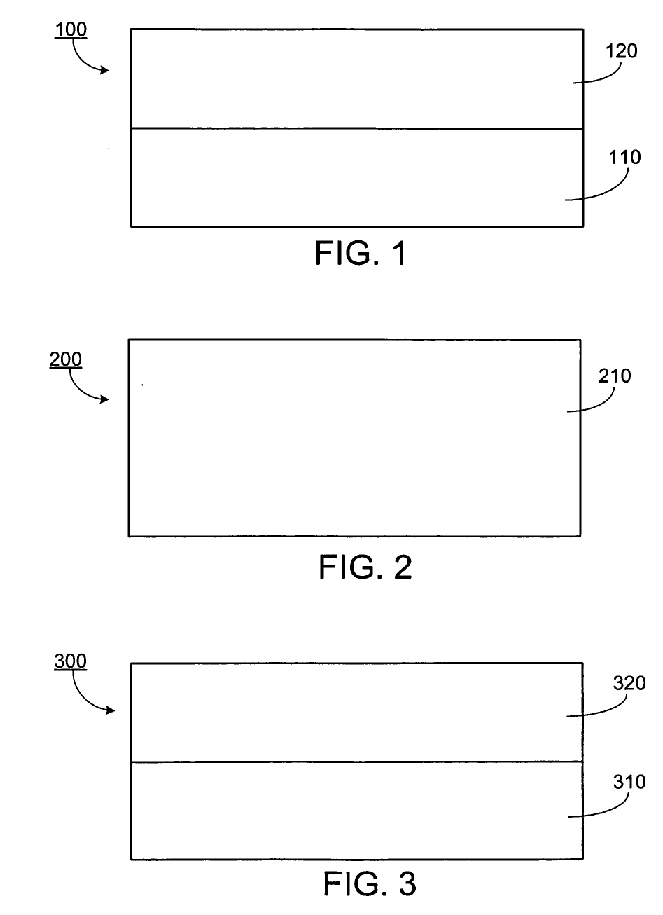

As an example, FIG. 1 shows an article 100 including a substrate 110 formed of a biocompatible material and a layer 120 of an antimicrobial material 120 disposed on substrate 110. In general, in such embodiments, the article can include any desired amount of antimicrobial material. For example, at least about 0.5 weight percent (e.g., at least about one weight percent, at least about three weight percent, at least about four weight percent, at least about five weigh percent, at least about 10 weight percent, at least about 20 weight percent) of the article can be formed of the antimicrobial material, and/or at most about 95 weight percent (e.g., at most about 90 weight percent, at most about 80 weight percent, at most about 60 weight percent, at most about 50 weight percent) of the article can be formed of the antimicrobial material.

As another example, FIG. 2 shows an article 200 including a substrate 210 formed of a biocompatible material and an antimicrobial material disposed in the biocompatible material. Generally, in such embodiments, the article can include any desired amount of antimicrobial material. For example, at least about 0.5 weight percent (e.g., at least about one weight percent, at least about three weight percent, at least about four weight percent, at least about five weigh percent, at least about 10 weight percent, at least about 20 weight percent) of the article can be formed of the antimicrobial material, and/or at most about 95 weight percent (e.g., at most about 90 weight percent, at most about 80 weight percent, at most about 60 weight percent, at most about 50 weight percent) of the article can be formed of the antimicrobial material.

As a further example, FIG. 3 shows an article 300 including a substrate 310 formed of a biocompatible material and an antimicrobial material disposed in the biocompatible material, and a layer 310 of antimicrobial material disposed on substrate 310. In general, in such embodiments, the article can include any desired amount of antimicrobial material. For example, at least about 0.5 weight percent (e.g., at least about one weight percent, at least about three weight percent, at least about four weight percent, at least about five weigh percent, at least about 10 weight percent, at least about 20 weight percent) of the article can be formed of the antimicrobial material, and/or at most about 95 weight percent (e.g., at most about 90 weight percent, at most about 80 weight percent, at most about 60 weight percent, at most about 50 weight percent) of the article can be formed of the antimicrobial material. In some embodiments, at least about 0.5 weight percent (e.g., at least about one weight percent, at least about three weight percent, at least about four weight percent, at least about five weigh percent, at least about 10 weight percent, at least about 20 weight percent) of the article can be formed of the antimicrobial material disposed in the substrate, and/or at most about 95 weight percent (e.g., at most about 90 weight percent, at most about 80 weight percent, at most about 60 weight percent, at most about 50 weight percent) of the article can be formed of the antimicrobial material disposed in the substrate. In certain embodiments, at least about 0.5 weight percent (e.g., at least about one weight percent, at least about three weight percent, at least about four weight percent, at least about five weigh percent, at least about 10 weight percent, at least about 20 weight percent) of the article can be formed of the antimicrobial material disposed in the layer supported by the substrate, and/or at most about 95 weight percent (e.g., at most about 90 weight percent, at most about 80 weight percent, at most about 60 weight percent, at most about 50 weight percent) of the article can be formed of the antimicrobial material disposed in the layer supported by the substrate.

In certain embodiments, the article is in the form of a paste or a gel (e.g., when used as a tissue bulking agent), a semi-solid, a nano-dispersed suspension, and/or a colloidal dispersion. Generally, in such embodiments, the article can include any desired amount of antimicrobial material. For example, at least about 0.5 weight percent (e.g., at least about one weight percent, at least about three weight percent, at least about four weight percent, at least about five weigh percent, at least about 10 weight percent, at least about 20 weight percent) of the article can be formed of the antimicrobial material, and/or at most about 95 weight percent (e.g., at most about 90 weight percent, at most about 80 weight percent, at most about 60 weight percent, at most about 50 weight percent) of the article can be formed of the antimicrobial material.

Typically, the biocompatible material is selected based on the intended use of the article. As an example, in some embodiments in which the article is intended to be used as an implant, the biocompatible material can be in the form of a substrate (e.g., when used as an implant), and the antimicrobial material can, for example, be supported by (e.g., disposed on) the substrate. As another example, in certain embodiments in which the article is intended to be used as a tissue bulking agent, a tissue reinforcing agent, and/or a tissue suspending agent, the article can be in the form of a paste, and the antimicrobial material can, for example, be mixed with the biocompatible material.

In some embodiments, the biocompatible material is a naturally occurring biocompatible material. For example, the biocompatible material can be derived (e.g., in the form of tissue) from a human or an animal (e.g., a pig, a dog, a cat, a horse, a bird, a reptile, an amphibian, a fish, a turtle, a guinea pig, a hamster, a rodent, a cow, a goat, a primate, a monkey, a chicken, a turkey, a buffalo, an ostrich, a sheep, a llama). In some embodiments, the biocompatible material is cultured. In certain embodiments, the biocompatible material is derived from the subject. Examples of such materials include collagen, skin, heart valve, ligament, tendon and/or muscle. Examples of such materials include Permacol™ Collagen Implant (Tissue Science Laboratories, Covington, Ga.), Carticel (Genzyme), collagen (Collagen Corporation), hyaluronic acid (Synvisc (Genzyme Corporation), Restalyne (Medicis)). In certain embodiments, the biocompatible material is a synthetic material. For example, the biocompatible material can be polypropylene, polylactic acid (PLA), a polyester, a plastic, a poly-foam or polygalactic acid (PGA). In general, any appropriate antimicrobial material can be used.

In some embodiments, a metal-containing antimicrobial material can be used.

Examples of metals that can be contained in a metal-containing antimicrobial materials include silver, gold, platinum, palladium, iridium, copper, tin, antimony and bismuth.

Examples of metal-containing antimicrobial materials include metal nitrates, metal sulfadiazines, metal carbonates, metal acetates, metal lactates, metal citrates, metal oxides, metal hydroxides, metal succinates, metal chlorates, metal stearates, metal sorbates, metal oleates, metal glutonates, metal adipates, metal myristates, and alkali metal thiosulphates.

Examples of antimicrobial metal-containing materials (which may or may not also be an atomically disordered crystalline material or a nanocrystalline material) include antimicrobial silver-containing materials (e.g., antimicrobial silver, antimicrobial silver alloys, antimicrobial silver oxides, antimicrobial silver carbides, antimicrobial silver nitrides, antimicrobial silver borides, antimicrobial silver sulfides, antimicrobial silver myristates, antimicrobial silver stearates, antimicrobial silver oleates, antimicrobial silver glutonates, antimicrobial silver adipates, antimicrobial silver silicates, antimicrobial silver phosphides, antimicrobial silver halides, antimicrobial silver hydrides, antimicrobial silver nitrates, antimicrobial silver carbonates, antimicrobial silver sulfadiazines, antimicrobial silver acetates, antimicrobial silver lactates, antimicrobial silver citrates, antimicrobial alkali silver thiosulphates (e.g., antimicrobial sodium silver thiosulphate, antimicrobial potassium silver thiosulphate)), antimicrobial gold-containing materials (e.g., antimicrobial gold, antimicrobial gold alloys, antimicrobial gold oxides, antimicrobial gold carbides, antimicrobial gold nitrides, antimicrobial gold borides, antimicrobial gold sulfides, antimicrobial gold myristates, antimicrobial gold stearates, antimicrobial gold oleates, antimicrobial gold glutonates, antimicrobial gold glutonates, antimicrobial gold adipates, antimicrobial gold silicates, antimicrobial gold phosphides, antimicrobial gold halides, antimicrobial gold hydrides, antimicrobial gold nitrates, antimicrobial gold carbonates, antimicrobial gold sulfadiazines, antimicrobial gold acetates, antimicrobial gold lactates, antimicrobial gold citrates, antimicrobial alkali gold thiosulphates (e.g., antimicrobial sodium gold thiosulphate, antimicrobial potassium gold thiosulphate)), antimicrobial platinum-containing materials (e.g., antimicrobial platinum, antimicrobial platinum alloys, antimicrobial platinum oxides, antimicrobial platinum carbides, antimicrobial platinum nitrides, antimicrobial platinum borides, antimicrobial platinum sulfides, antimicrobial platinum myristates, antimicrobial platinum stearates, antimicrobial platinum oleates, antimicrobial platinum glutonates, antimicrobial platinum glutonates, antimicrobial platinum adipates, antimicrobial platinum silicates, antimicrobial platinum phosphides, antimicrobial platinum halides, antimicrobial platinum hydrides, antimicrobial platinum nitrates, antimicrobial platinum carbonates, antimicrobial platinum sulfadiazines, antimicrobial platinum acetates, antimicrobial platinum lactates, antimicrobial platinum citrates, antimicrobial alkali platinum thiosulphates (e.g., antimicrobial sodium platinum thiosulphate, antimicrobial potassium platinum thiosulphate)), antimicrobial palladium-containing materials (e.g., antimicrobial palladium, antimicrobial palladium alloys, antimicrobial palladium oxides, antimicrobial palladium carbides, antimicrobial palladium nitrides, antimicrobial palladium borides, antimicrobial palladium sulfides, antimicrobial palladium myristates, antimicrobial palladium stearates, antimicrobial palladium oleates, antimicrobial palladium glutonates, antimicrobial palladium glutonates, antimicrobial palladium adipates, antimicrobial palladium silicates, antimicrobial palladium phosphides, antimicrobial palladium halides, antimicrobial palladium hydrides, antimicrobial palladium nitrates, antimicrobial palladium carbonates, antimicrobial palladium sulfadiazines, antimicrobial palladium acetates, antimicrobial palladium lactates, antimicrobial palladium citrates, antimicrobial alkali palladium thiosulphates (e.g., antimicrobial sodium palladium thiosulphate, antimicrobial potassium palladium thiosulphate)), antimicrobial iridium-containing materials (e.g., antimicrobial iridium, antimicrobial iridium alloys, antimicrobial iridium oxides, antimicrobial iridium carbides, antimicrobial iridium nitrides, antimicrobial iridium borides, antimicrobial iridium sulfides, antimicrobial iridium myristates, antimicrobial iridium stearates, antimicrobial iridium oleates, antimicrobial iridium glutonates, antimicrobial iridium glutonates, antimicrobial iridium adipates, antimicrobial iridium silicates, antimicrobial iridium phosphides, antimicrobial iridium halides, antimicrobial iridium hydrides, antimicrobial iridium nitrates, antimicrobial iridium carbonates, antimicrobial iridium sulfides, antimicrobial iridium sulfadiazines, antimicrobial iridium acetates, antimicrobial iridium lactates, antimicrobial iridium citrates, antimicrobial alkali iridium thiosulphates (e.g., antimicrobial sodium iridium thiosulphate, antimicrobial potassium iridium thiosulphate)), antimicrobial zinc-containing materials (e.g., antimicrobial zinc, antimicrobial zinc alloys, antimicrobial zinc oxides, antimicrobial zinc carbides, antimicrobial zinc nitrides, antimicrobial zinc borides, antimicrobial zinc sulfides, antimicrobial zinc myristates, antimicrobial zinc stearates, antimicrobial zinc oleates, antimicrobial zinc glutonates, antimicrobial zinc glutonates, antimicrobial zinc adipates, antimicrobial zinc silicates, antimicrobial zinc phosphides, antimicrobial zinc halides, antimicrobial zinc hydrides, antimicrobial zinc nitrates, antimicrobial zinc carbonates, antimicrobial zinc sulfides, antimicrobial zinc sulfadiazines, antimicrobial zinc acetates, antimicrobial zinc lactates, antimicrobial zinc citrates, antimicrobial alkali zinc thiosulphates (e.g., antimicrobial sodium zinc thiosulphate, antimicrobial potassium zinc thiosulphate)), antimicrobial copper-containing materials (e.g., antimicrobial copper, antimicrobial copper alloys, antimicrobial copper oxides, antimicrobial copper carbides, antimicrobial copper nitrides, antimicrobial copper borides, antimicrobial copper sulfides, antimicrobial copper myristates, antimicrobial copper stearates, antimicrobial copper oleates, antimicrobial copper glutonates, antimicrobial copper glutonates, antimicrobial copper adipates, antimicrobial copper silicates, antimicrobial copper phosphides, antimicrobial copper halides, antimicrobial copper hydrides, antimicrobial copper nitrates, antimicrobial copper carbonates, antimicrobial copper sulfides, antimicrobial copper sulfadiazines, antimicrobial copper acetates, antimicrobial copper lactates, antimicrobial copper citrates, antimicrobial alkali copper thiosulphates (e.g., antimicrobial sodium copper thiosulphate, antimicrobial potassium copper thiosulphate)), antimicrobial tin-containing materials (e.g., antimicrobial tin, antimicrobial tin alloys, antimicrobial tin oxides, antimicrobial tin carbides, antimicrobial tin nitrides, antimicrobial tin borides, antimicrobial tin sulfides, antimicrobial tin myristates, antimicrobial tin stearates, antimicrobial tin oleates, antimicrobial tin glutonates, antimicrobial tin glutonates, antimicrobial tin adipates, antimicrobial tin silicates, antimicrobial tin phosphides, antimicrobial tin halides, antimicrobial tin hydrides, antimicrobial tin nitrates, antimicrobial tin carbonates, antimicrobial tin sulfides, antimicrobial tin sulfadiazines, antimicrobial tin acetates, antimicrobial tin lactates, antimicrobial tin citrates, antimicrobial alkali tin thiosulphates (e.g., antimicrobial sodium tin thiosulphate, antimicrobial potassium tin thiosulphate)), antimicrobial antimony-containing materials (e.g., antimicrobial antimony, antimicrobial antimony alloys, antimicrobial antimony oxides, antimicrobial antimony carbides, antimicrobial antimony nitrides, antimicrobial antimony borides, antimicrobial antimony sulfides, antimicrobial antimony myristates, antimicrobial antimony stearates, antimicrobial antimony oleates, antimicrobial antimony glutonates, antimicrobial antimony glutonates, antimicrobial antimony adipates, antimicrobial antimony silicates, antimicrobial antimony phosphides, antimicrobial antimony halides, antimicrobial antimony hydrides, antimicrobial antimony nitrates, antimicrobial antimony carbonates, antimicrobial antimony sulfides, antimicrobial antimony sulfadiazines, antimicrobial antimony acetates, antimicrobial antimony lactates, antimicrobial antimony citrates, antimicrobial alkali antimony thiosulphates (e.g., antimicrobial sodium antimony thiosulphate, antimicrobial potassium antimony thiosulphate)), antimicrobial bismuth containing materials (e.g., antimicrobial bismuth, antimicrobial bismuth alloys, antimicrobial bismuth oxides, antimicrobial bismuth carbides, antimicrobial bismuth nitrides, antimicrobial bismuth borides, antimicrobial bismuth sulfides, antimicrobial bismuth myristates, antimicrobial bismuth stearates, antimicrobial bismuth oleates, antimicrobial bismuth glutonates, antimicrobial bismuth glutonates, antimicrobial bismuth adipates, antimicrobial bismuth silicates, antimicrobial bismuth phosphides, antimicrobial bismuth halides, antimicrobial bismuth hydrides, antimicrobial bismuth nitrates, antimicrobial bismuth carbonates, antimicrobial bismuth sulfides, antimicrobial bismuth sulfadiazines, antimicrobial bismuth acetates, antimicrobial bismuth lactates, antimicrobial bismuth citrates, antimicrobial alkali bismuth thiosulphates (e.g., antimicrobial sodium bismuth thiosulphate, antimicrobial potassium bismuth thiosulphate)).

In some embodiments, a metal-containing antimicrobial material (e.g., one or more of the above-listed antimicrobial metals) can be a nanocrystalline material. In certain embodiments, a metal-containing antimicrobial material can be an atomically disordered, crystalline metal-containing (e.g., silver-containing) material. In some embodiments, the material can be an atomically disordered, nanocrystalline metal-containing (e.g., silver-containing) material. Such materials and their methods of manufacture are disclosed, for example, in co-pending and commonly owned U.S. Ser. No. 10/690,774, filed Oct. 22, 2003, and entitled “Metal-Containing Materials”, and co-pending and commonly owned U.S. Ser. No. 11/070,903, filed Mar. 3, 2005, and entitled “NO-Containing Complexes”, both of which are hereby incorporated by reference.

Additional examples of antimicrobial materials include antibiotic materials, antifungal materials, antiviral materials, antiseptics (e.g., povidone iodine, chlorhexidines, quaternary ammonium compounds, glutaraldehydes, alcohols, and chlorine-containing compounds) and iodine.

Generally, an article can be formed as desired.

In some embodiments, an antimicrobial coating (e.g., Silcryst™, NUCRYST Pharmaceuticals, Wakefield, Mass.) can be applied to a biocompatible substrate (e.g., a biodegradable thread, a biodegradable string, a thread woven into an implant, a string woven into an implant).

In certain embodiments, an implant can include a substrate (e.g., a tissue-derived substrate, such as a collagen substrate) an a coating of a powder of antimicrobial material (e.g., silver powder). Such an implant can be formed, for example, by moistening the substrate, placing the substrate into a volume of powder, and allowing the powder to adhere to the moistened substrate (e.g., prior to insertion into the subject). This may be achieved by rolling the moistened substrate in powder or by placing the moistened substrate in a bag containing the powder and shaking the bag to uniformly coat the device. Other such approaches of adhering the powder to the implant may also be used. Such approaches can include, for example, vapor depositing (e.g., chemical vapor deposition, physical vapor deposition), homogenizing, and/or physically embedding. In some embodiments, the antimicrobial material can be sputtered onto the biocompatible material.

In some embodiments (e.g., when the substrate is in the form of a medical device, such as a catheter, a valve, a vessel, a cornea, a bone or a tooth) an antimicrobial material (e.g., silver) can be mirrored onto the substrate. Mirroring refers to plating a metal (e.g., silver metal [Ag(0)]) onto a surface, by exposing the surface to metal ions (e.g., silver ions [Ag+]) and then electronically reducing the metal ions (e.g., silver ions) to metal (e.g., silver metal).

In embodiments in which the article is an implant and the tissue-derived biocompatible material used that is in the form of a slurry, the antimicrobial material can be mixed with the slurry before the slurry is formed into the implant.

In certain embodiments, the methods can include chemically reducing a tissue-derived biocompatible material (e.g., Permacol™ Collagen Implant) or other tissue derived implant, adding an antimicrobial material (e.g., nanocrystalline silver), and then oxidizing (e.g., slowly oxidizing) the mixture to covalently fix the silver to the biocompatible material.

In some embodiments, the methods can include soaking the biocompatible material (e.g., Permacol™ Collagen Implant) in a solution (e.g., an isotonic metal, such as silver, solution) or a metal (e.g., silver) colloidal dispersion, and using the solution or colloidal dispersion to store the product until use.

In embodiments in which a solution or dispersion containing the antimicrobial material is used, the solution or dispersion can generally contain any desired amount of the antimicrobial material. For example, in some embodiments, the solution or dispersion contains at least about 0.5 weight percent (e.g., at least about one weight percent, at least about three weight percent, at least about four weight percent, at least about five weigh percent, at least about 10 weight percent, at least about 20 weight percent) of the antimicrobial material, and/or at most about 95 weight percent (e.g., at most about 90 weight percent, at most about 80 weight percent, at most about 60 weight percent, at most about 50 weight percent) of the antimicrobial material.

In certain embodiments, a solution or dispersion can include one or more additional species. For example, in certain embodiments, a solution or dispersion may include one or more excipients (e.g., PVA, a lecithin, a citrate, a lactate, ater, carbon dioxide).

In certain embodiments, the methods can include soaking the biocompatible material (e.g., tissue-derived biocompatible material) in a hypotonic metal (e.g., silver) solution or a metal (e.g., silver) colloidal dispersion (so that the biocompatible material will swell) until the metal has penetrated into the biocompatible material, followed by bringing the biocompatible material to an isotonic system (e.g., to allow the biocompatible material to shrink back to its original size). This may embed the antimicrobial material in the biocompatible material, which can allow for the release of the antimicrobial material from the biocompatible material to be controlled (e.g., by diffusion of the antimicrobial material, by erosion of the biocompatible material).

In some embodiments, the methods can include reductive coating of the biocompatible material (e.g., Permacol™ Collagen Implant) with the antimicrobial material. This may enhance the preservation of the scaffold and/or result in a thin film/mirror film of (e.g., of silver) on the surface.

Optionally ion-exchange therapeutic resins, involving exchange of silver ions for other ions, can be used. For example, in some embodiments, the methods include binding a metal (e.g., silver) to a biocompatible material (e.g., porcine skin) by ion exchange, and allowing proteolytic activity in a wound to enzymatically release the antimicrobial material (e.g., silver) gradually as the skin is degraded. In some embodiments, an antimicrobial material (e.g., a metal-containing materials, such as a silver-containing material) can be ion-exchanged onto other types of molecular scaffold such as derivatized dextran, hyaluronic acid, and/or glycogen.

In certain embodiments, the methods can include making a metal-collagen (e.g., silver-collagen) solution or gel and coating the collagen sheet or other tissue derived material with the silver-collagen gel. The coating can be cross-linked to make the coating, thus silver activity, last longer.

In some embodiments, the methods include removing a product (e.g., a Permacol™ Collagen Implant -based product) from its packaging and dipping the product into a solution containing an antimicrobial material (e.g., nanocrystalline silver).

In certain embodiments, the methods include making the edges of the biocompatible material bordered with a metal (e.g., silver) embedded material (e.g., a gauze-like substance). In some embodiments, an antimicrobial (e.g., silver) thread can run through the edge of the biocompatible material.

In some embodiments, the biocompatible material can be sandwiched between two pieces (e.g., of equal size) of antimicrobial material (e.g., Acticoat® dressing) and can be stored in a pouch with suitable buffer for continuous release of silver from the dressing to the biocompatible material (e.g., to maintain antimicrobial activity).

Optionally, the methods can include sonication.

While certain embodiments have been disclosed, other embodiments are possible.

As an example, while embodiments have been described in which a single antimicrobial material is used, in certain embodiments, multiple antimicrobial materials can be used.

As an example, while certain metal-containing antimicrobial materials have been disclosed, more generally, any metal-containing materials may also be used (e.g., one of the metal-containing materials noted above in a non-antimicrobial form). In some embodiments, one or more such materials can be used, alone or in combination with one or more other antimicrobial materials.

As another example, while antimicrobial materials have been described, other materials may be used. Examples of such materials include anti-MMP materials, anti-inflammatory materials, and pro-healing growth factors. In some embodiments, an antimicrobial material may also be an anti-inflammatory and/or anti-MMP material. For example, in certain embodiments, one of the above-noted antimicrobial metal-containing materials (e.g. a silver-containing material, such as a nanocrystalline silver containing material) may also be an anti-inflammatory material and/or an anti-MMP material. In some embodiments, one or more such materials can be used, alone or in combination with one or more other antimicrobial materials.

Other embodiments are in the claims.

1. An article, comprising:
a biocompatible material; and
an antimicrobial material associated with the biocompatible material,
wherein the biocompatible material is a naturally occurring biocompatible material.

2. The article of claim 1, wherein the biocompatible material is a substrate.

3. The article of claim 2, wherein at least a first portion of the antimicrobial material comprises a coating on the substrate.

4. The article of claim 3, wherein a second portion of the antimicrobial material is mixed with the biocompatible material.

5. The article of claim 2, wherein at least a portion of the antimicrobial material is mixed with the biocompatible material.

6. The article of claim 1, wherein the biocompatible material and the antimicrobial material form a mixture.

7. The article of claim 6, wherein the mixture is in the form of a paste or gel.

8. The article of claim 1, wherein the article is in the form of a paste or a gel.

9. The article of claim 1, wherein the biocompatible material is derived from a human or an animal.

10. The article of claim 9, wherein the biocompatible material is cultured.

11. The article of claim 9, wherein the animal is selected from the group consisting of a pig, a dog, a cat, a horse, a bird, a reptile, an amphibian, a fish, a turtle, a guinea pig, a hamster, a rodent, a cow, a goat, a primate, a monkey, a chicken, a turkey, a buffalo, an ostrich, a sheep, a llama.

12. The article of claim 9, wherein the biocompatible material comprises tissue from the human or the animal.

13. The article of claim 1, wherein the antimicrobial material comprises an antibiotic material, an antifungal material or an antiviral material.

14. The article of claim 1, wherein the antimicrobial material comprises at least one metal.

15. The article of claim 14, wherein the at least one metal is selected from the group consisting of silver, gold, platinum, palladium, iridium, copper, tin, antimony and bismuth.

16. The article of claim 14, wherein the at least one metal is selected from the group consisting of silver, gold, platinum and palladium.

17. The article of claim 14, wherein the at least one metal comprises silver.

18. The article of claim 14, wherein the antimicrobial material comprises at least one material selected from the group consisting of metal nitrates, metal sulfadiazines, metal carbonates, metal acetates, metal lactates, metal citrates, metal oxides, metal hydroxides, metal succinates, metal chlorates, metal stearates, metal sorbates, metal oleates, metal glutonates, metal adipates, metal myristates, and alkali metal thiosulphates.

19. The article of claim 14, wherein the antimicrobial material comprises at least one material selected from the group consisting of silver nitrates, silver sulfadiazines, silver carbonates, silver acetates, silver lactates, silver citrates, silver oxides, silver hydroxides, silver succinates, silver chlorates, silver stearates, silver sorbates, silver oleates, silver glutonates, silver adipates, silver myristates, and alkali silver thiosulphates.

20. The article of claim 14, wherein the antimicrobial material comprises a nanocrystalline material.

21. The article of claim 20, wherein the antimicrobial material comprises a material that is an atomically disordered, crystalline material.

22. The article of claim 14, wherein the antimicrobial material comprises a material that is an atomically disordered, crystalline material.

23. The article of claim 14, wherein the antimicrobial material comprises a nanocrystalline silver-containing material.

24. The article of claim 23, wherein the antimicrobial material comprises a material that is an atomically disordered, crystalline silver-containing material.

25. The article of claim 14, wherein the antimicrobial material comprises a material that is an atomically disordered, crystalline silver-containing material.

26. The article of claim 1, wherein the article is a wound dressing or a wound packing.

27. The article of claim 1, wherein the article is configured for use in a subject.

28. The article of claim 27, wherein the article is configured for use as a tissue bulking agent, a tissue reinforcing agent, or a tissue suspending agent.

29. The article of claim 27, wherein the article is configured for use as an implant.

30.-76. (canceled)
